(19) United States
(12) Patent Application Publication
(10) Pub. No.: US 2017/0150927 A1
(43) Pub. Date: Jun. 1, 2017

(54) ADHESIVE PLASTER MODULE

(71) Applicants: SHINKO ELECTRIC INDUSTRIES CO., LTD., Nagano (JP); NIHON KOHDEN CORPORATION, Tokyo (JP)

(72) Inventors: Kazuyuki KUBOTA, Nagano (JP); Kaori YOKOTA, Nagano (JP); Norihito KONNO, Tokyo (JP)

(21) Appl. No.: 15/338,534

(22) Filed: Oct. 31, 2016

(30) Foreign Application Priority Data
Dec. 1, 2015 (JP) 2015-234809

(51) Int. Cl. A61B 5/00; A61B 5/145; A61B 5/029; A61B 5/0402; H01M 2/10; A61B 5/0205

(52) U.S. Cl. CPC A61B 5/6833; H01M 2/1038; A61B 5/02055; A61B 5/029; A61B 5/0402; A61B 5/14532; A61B 5/14542; H01M 2220/30; A61B 5/02438

(57) ABSTRACT

An adhesive plaster module which has an adhesive layer for adhering to an object includes a substrate having the adhesive layer, a switch mounted on a same side of the substrate as the adhesive layer, a battery mounted on an opposite side of the substrate from the adhesive layer, and an electronic component mounted on the opposite side of the substrate from the adhesive layer, and configured to operate with power supplied from the battery upon the switch becoming conductive, wherein the switch is configured to be nonconductive while the adhesive layer is not attached to the object, and configured to become conductive in response to attaching the adhesive layer to the object.

### CROSS-REFERENCE TO RELATED APPLICATIONS

The present application is based upon and claims the benefit of priority from the prior Japanese Patent Application No. 2015-234809 filed on Dec. 1, 2015, with the Japanese Patent Office, the entire contents of which are incorporated herein by reference.

### FIELD

The disclosures herein relate to an adhesive plaster module.

### BACKGROUND

Medical-purpose modules capable of adhering to an object such as human skin are known in the art (see Patent Document 1, for example). An adhesive plaster module, which is one of such modules, has one face serving as an adhesive face, which is attached to an object when used.

An adhesive plaster module may have a battery and an electronic component operating with electric power supplied from the battery. A switch may be disposed on the face opposite the adhesive face to disconnect power supply from the battery to the electronic component for the purpose of avoiding needless power consumption during nonuse.

The switch is tuned off when the adhesive plaster module is not used. The switch is turned on when the adhesive plaster module is used upon being attached to an object, thereby causing the battery to supply power to the electronic component to operate the electronic component.

The related-art adhesive plaster module having the switch requires two actions, i.e., attaching the adhesive plaster module to an object and turning on the switch to activate the power.

Such requirements lower the ease of use.

[Patent Document 1] Japanese Laid-open Patent Publication No. H2-26568

### SUMMARY

According to an aspect of an embodiment, an adhesive plaster module which has an adhesive layer for adhering to an object includes a substrate having the adhesive layer, a switch mounted on a same side of the substrate as the adhesive layer, a battery mounted on an opposite side of the substrate from the adhesive layer, and an electronic component mounted on the opposite side of the substrate from the adhesive layer, and configured to operate with power supplied from the battery upon the switch becoming conductive, wherein the switch is configured to be nonconductive while the adhesive layer is not attached to the object, and configured to become conductive in response to attaching the adhesive layer to the object.

The object and advantages of the embodiment will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIGS. 1A through 1C are drawings illustrating an example of an adhesive plaster module of a first embodiment;

FIG. 2 is a cross-sectional view illustrating an example of the way the adhesive plaster module of the first embodiment is used;

FIG. 3 is a cross-sectional view illustrating an example of the adhesive plaster module of the first embodiment that is packaged;

FIG. 4 is a cross-sectional view illustrating an example of an adhesive plaster module of a second embodiment;

FIG. 5 is a cross-sectional view illustrating an example of the way the adhesive plaster module of the second embodiment is used;

FIG. 6 is a cross-sectional view illustrating an example of the adhesive plaster module of the second embodiment that is packaged;

FIG. 7 is a cross-sectional view illustrating an example of an adhesive plaster module of a third embodiment;

FIG. 8 is a cross-sectional view illustrating an example of the way the adhesive plaster module of the third embodiment is used;

FIG. 9 is a cross-sectional view illustrating an example of the adhesive plaster module of the third embodiment that is packaged; and

FIGS. 10A and 10B are cross-sectional views illustrating the removal of packaging materials.

### DESCRIPTION OF EMBODIMENTS

In the following, embodiments will be described by referring to the accompanying drawings. In these drawings, the same elements are referred to by the same references, and a duplicate description thereof may be omitted.

### First Embodiment

### [Structure of Adhesive Plaster Module of First Embodiment]

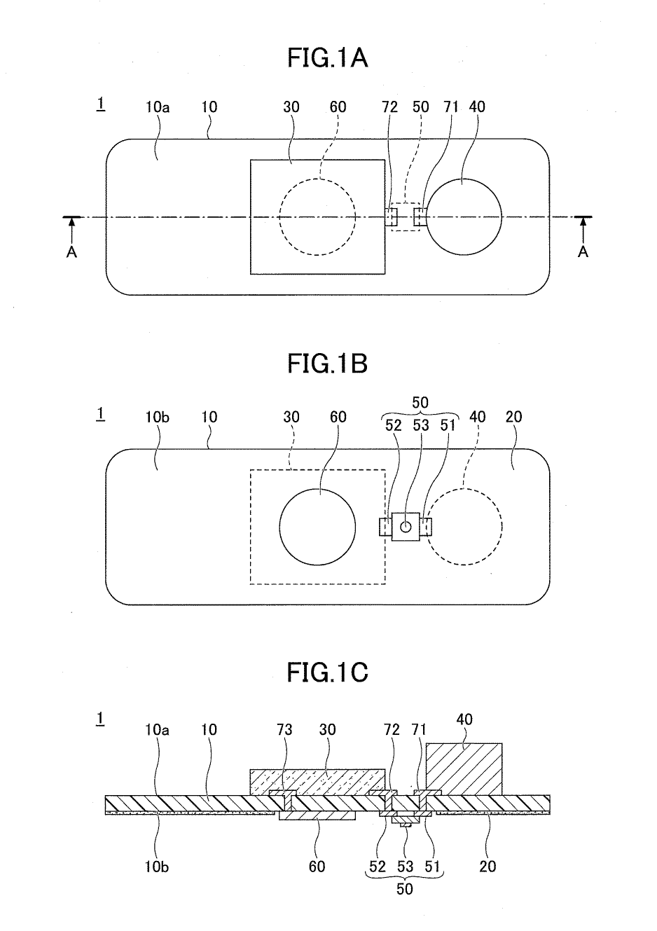

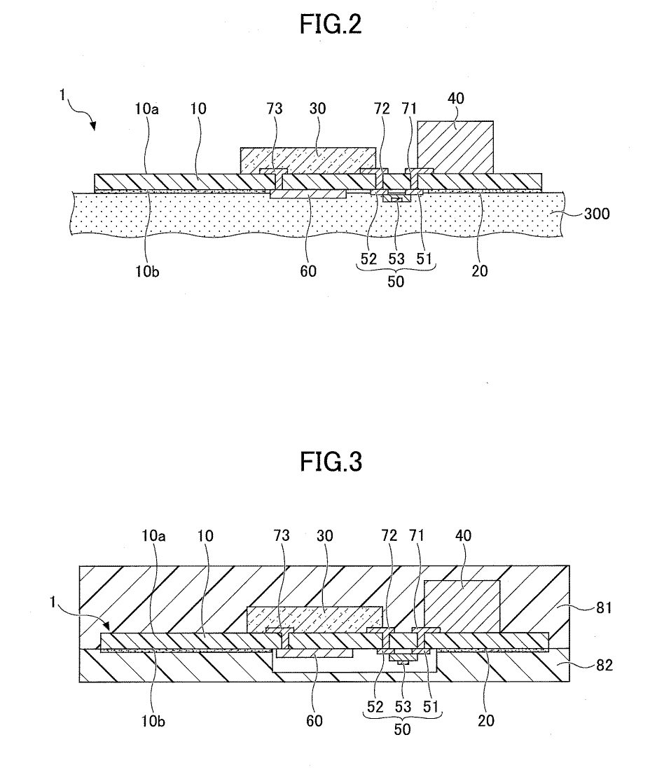

In the following, a description will be first given of the structure of an adhesive plaster module according to a first embodiment. FIGS. 1A through 1C are drawings illustrating an example of the adhesive plaster module according to the first embodiment. FIG. 1A illustrates a plan view, and FIG. 1B illustrates a bottom view, with FIG. 1C illustrating a cross-sectional view taken along a line A-A in FIG. 1A. FIG. 2 is a cross-sectional view illustrating an example of the way the adhesive plaster module of the first embodiment is used. FIG. 3 is a cross-sectional view illustrating an example of the adhesive plaster module of the first embodiment that is packaged.

In FIGS. 1A through 1C, an adhesive plaster module 1 includes a substrate 10, an adhesive layer 20, an electronic component 30, a battery 40, a switch 50, an electrode 60, and interconnections 71 through 73.

The adhesive plaster module 1 is attached to an object to acquire biological information from the object. The adhesive plaster module 1 may include a memory, which may store the acquired information. The adhesive plaster module 1 may include a wireless transmission device, which may transmit the acquired information through radio waves to an external device. The object may be a human body (i.e., subject), for example. The adhesive plaster module 1 may monitor the biological information of the subject. Examples of biological information include bioelectric potential, body temperature, heart rate, arterial blood oxygen saturation, blood glucose level, etc. The monitoring of bioelectric potential noted herein broadly covers any acquisition of a change in an electric potential relating to biological information, and may include acquisition of electrocardiogram, respiratory system impedance, thermistor respiratory monitor, heartbeat, cardiac output, etc.

In the present embodiment, for the sake of convenience, the side toward which the electronic component 30 of the adhesive plaster module 1 faces is referred to as an upper side or a first side, and the side toward which the switch 50 faces is referred to as a lower side or a second side. A surface (or face) of a given member that faces toward the upper side is referred to as a first surface (or first face) or an upper surface (or upper face), and a surface (or face) of the given member that faces toward the lower side is referred to as a second surface (or second face) or a lower surface (or lower face). It may be noted, however, that the adhesive plaster module 1 may be used in an upside-down position, or may be placed at any angle. Further, a plan view refers to a view taken in the direction normal to a first surface 10a of the substrate 10, and a plan shape is a shape as viewed in the direction normal to the first surface 10a of the substrate 10.

The substrate 10, which serves as a base body on which the electronic component 30, the battery 40, and the like are mounted, is an elongated, flexible, stretchable tape. The substrate 10 has the first surface 10a and a second surface 10b that is the surface opposite the first surface 10a. The substrate 10 may have a multilayered structure.

The substrate 10 may be made of material such as polyolefin, polypropylene, polyester, polyethylene, polyurethane, or the like. The size of the substrate 10 in a plan view is not limited to any size, and may be 10 to 30 mm in a transverse direction and 80 to 120 mm in a longitudinal direction, for example. The thickness of the substrate 10 may approximately be 25 to 75 micrometers, for example.

The second surface 10b of the substrate 10 has the adhesive layer 20 disposed thereon to serve as an adhesive surface. The adhesive plaster module 1 may be stuck to an object by the adhesive layer 20. The adhesive layer 20 may be made of acrylic adhesive, rubber adhesive, silicone adhesive, or the like.

The electronic component 30 is mounted on the first surface 10a of the substrate 10. The electronic component 30 may include a semiconductor device and a sensor (e.g., acceleration sensor, temperature sensor, etc.) for acquiring biological information from an object. The electronic component 30 may alternately be electrically connected to a dedicated sensor (not shown) that comes in physical contact with the object to acquire biological information, for the purpose of acquiring information relating to heartbeat, electrocardiogram, or the like. The semiconductor device may be a semiconductor memory for storing biological information acquired by the sensor, a wireless transmitter for transmitting biological information wirelessly to an external device, a CPU (central processing unit), and the like.

The battery 40 is mounted on the first surface 10a of the substrate 10. The battery 40 has a positive pole that is electrically connected through the interconnection 71 inclusive of a through wire interconnect to a first contact 51 of the switch 50, which is mounted on the second surface 10b of the substrate 10. A second contact 52 of the switch 50 is electrically connected to the positive power terminal of the electronic component 30 through the interconnection 72 inclusive of a through wire interconnect. The battery 40 has a negative pole that is electrically connected through a direct connection interconnect (not shown) to the negative power terminal of the electronic component 30. Alternatively, the negative pole of the battery 40 may be electrically connected to the negative power terminal of the electronic component 30 through the switch 50. The battery 40 may be a button lithium battery, for example. A press-button switch may be used as the switch 50, for example.

In the case of the adhesive plaster module 1 being not attached to an object as illustrated in FIGS. 1A through 1C, a movable part 53 of the switch 50 is not pressed, thereby leaving the switch 50 in the off state, in which the first contact 51 and the second contact 52 are not electrically connected to each other (i.e., nonconductive). As a result, no power is supplied from the battery 40 to the electronic component 30.

In the case of the adhesive plaster module 1 being attached to an object 300, the movable part 53 of the switch 50 is pressed to place the switch 50 in the on state, in which the first contact 51 and the second contact 52 are electrically connected to each other. As a result, power is supplied from the battery 40 to the electronic component 30 through the interconnection 71, the switch 50, and the interconnection 72, thereby making the electronic component 30 operable.

The electrode 60 is formed on the second surface 10b of the substrate 10. As illustrated in FIG. 2, the electrode 60 is a sensing-purpose electrode that comes in physical contact with the object 300 when the adhesive plaster module 1 is attached to the object 300. The electrode 60 is electrically connected to the input terminal of the electronic component 30 through the interconnection 73 inclusive of a through wire interconnect, so that biological information is supplied from the object 300 to the electronic component 30 through the electrode 60 and the interconnection 73.

Cupper (Cu) or the like may be used as the material of the interconnections 71 through 73. The thickness of the interconnections 71 through 73 may approximately be 10 to 40 micrometers, for example. One or more patterns may be disposed in addition to the interconnections 71 through 73 according to need. Routing of the interconnections 71 through 73 may be performed by use of any routing patterns that are appropriate. Multilayered interconnections may be utilized, so that interconnections may be connected to each other through via interconnects as appropriate.

The object 300 may be a human body, in which case the adhesive plaster module 1 may be attached to the surface of the skin of the human body. For monitoring heartbeat, for example, the adhesive plaster module 1 is attached to an arm. For acquiring electrocardiogram signals, the adhesive plaster module 1 is attached to a chest. With such arrangements, biological information is monitored by use of the adhesive plaster module 1.

As illustrated in FIG. 3, an upper-side packaging material 81 and a lower-side packaging material 82 may cover the adhesive plaster module 1 for protection before use. In such a case, the adhesive plaster module 1 is removed from the upper-side packaging material 81 and the lower-side packaging material 82 before the adhesive plaster module 1 is attached to and used on an object. The upper-side packaging material 81 and the lower-side packaging material 82 may be made of insulating material such as resin.

As described above, the adhesive plaster module 1 includes the electronic component 30 and the battery 40 mounted on the first surface 10a of the substrate 10, and also includes the switch 50 mounted on the second surface 10b (i.e., on the same side as the adhesive layer) of the substrate 10. Sticking the adhesive layer 20 of the substrate 10 to an object causes the switch 50 to be turned on in conjunction with the sticking action, thereby allowing the battery 40 to supply electric power to the electronic component 30.

Unlike the related-art configuration, there is no need to perform two actions, i.e., attaching an adhesive plaster module to an object and operating the switch to activate the power. In the case of the adhesive plaster module 1, a single action of attaching the adhesive plaster module to an object causes simultaneous power activation, which improves the use of ease.

One type of related-art adhesive plaster modules provides a battery holder in the substrate so that inserting a battery into the battery holder causes power activation, and another type provides a paper sheet placed between a battery and an electrode disposed on the substrate so that removing the paper sheet causes power activation. Either of these types requires two actions, i.e., an action of attaching the adhesive plaster module to an object and an action of activating the power.

Even in comparison with these types of modules, the adhesive plaster module 1 is advantageous because it offers the improvement in the ease of use by allowing a single action to achieve both attachment to an object and activation of power.

### Second Embodiment

The second embodiment is directed to an adhesive plaster module that has a switch different from the switch used in the first embodiment. In connection with the second embodiment, a description of the same or similar constituent elements as those of the previously provided descriptions may be omitted as appropriate.

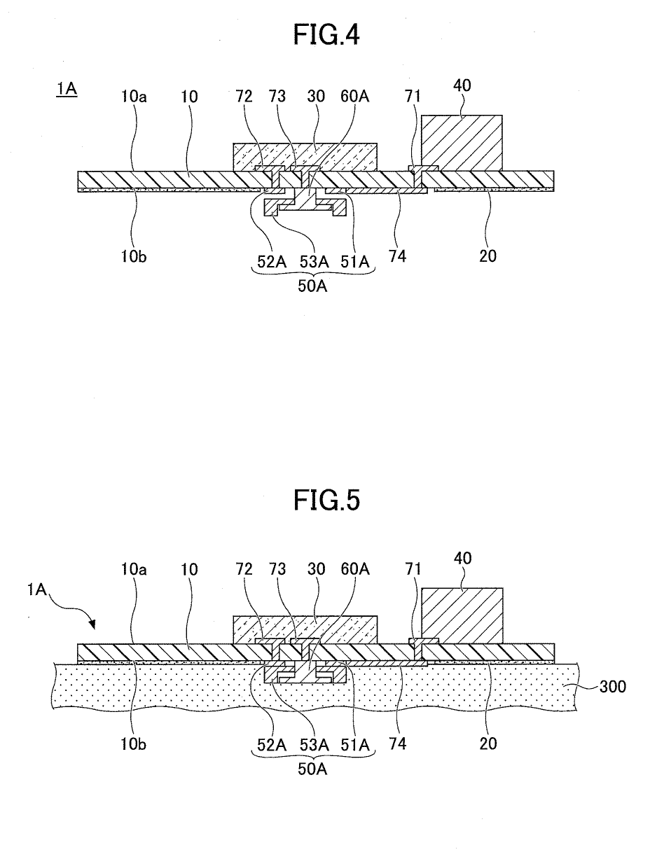

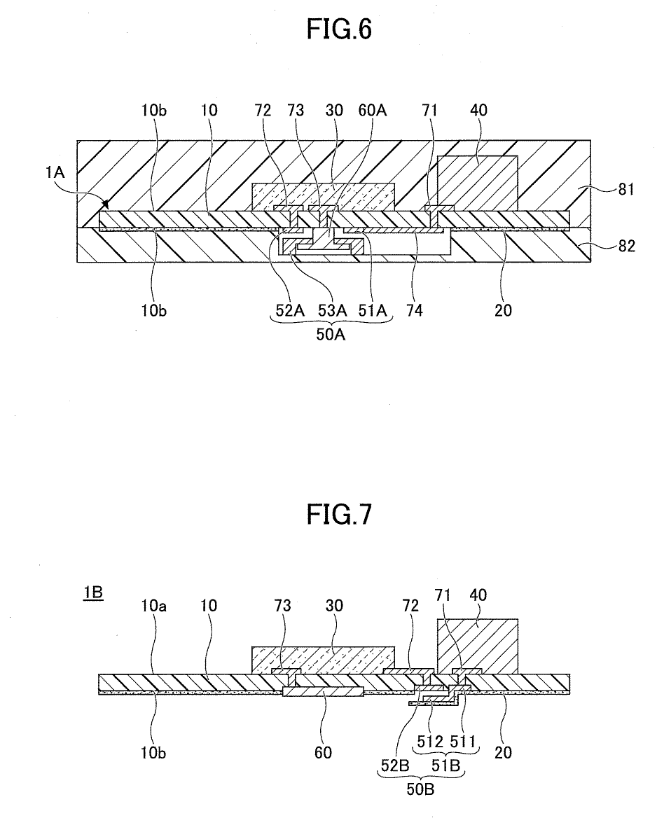

FIG. 4 is a cross-sectional view illustrating an example of an adhesive plaster module of the second embodiment. FIG. 5 is a cross-sectional view illustrating an example of the way the adhesive plaster module of the second embodiment is used. FIG. 6 is a cross-sectional view illustrating an example of the adhesive plaster module of the second embodiment that is packaged.

As illustrated in FIG. 4, an adhesive plaster module 1A of the second embodiment differs from the adhesive plaster module 1 (see FIGS. 1A through 1C, for example) in that a switch 50A is provided in place of the switch 50, and an electrode 60A is provided in place of the electrode 60.

The switch 50A includes a first contact 51A, a second contact 52A, and a movable part 53A. The first contact 51A, which is formed on the second surface 10b of the substrate 10, is electrically connected to the positive pole of the battery 40 through an interconnection 74 and the interconnection 71 inclusive of a through wire interconnect. The second contact 52A, which is formed on the second surface 10b of the substrate 10, is electrically connected to the positive power terminal of the electronic component 30 through the interconnection 72 inclusive of a through wire interconnect.

The movable part 53A has an annular shape that surrounds the periphery of the electrode 60A that has a circular columnar shape, for example, and has the perimeter rim thereof projecting in the same direction that the second surface 10b of the substrate 10 faces. The movable part 53A is movable in the upward and downward directions along the electrode 60 serving as a shaft. The lower face of the electrode 60A has a horizontally extended width, which serves as a stopper to prevent the movable part 53A from slipping off from the electrode 60A.

The movable part 53A may be made of metal such as titanium, aluminum, copper, or the like. It may be noted that the lower face and the inner wall surface of the movable part 53A are treated to have insulating property, so that the movable part 53A and the electrode 60A are insulated from each other. The upper face of the movable part 53A is not treated for insulation, and is an exposed metal face. The upper face of the movable part 53A may have a metal plating film made of gold or the like formed thereon.

In the case of the adhesive plaster module 1A being not attached to an object as illustrated in FIG. 4, the movable part 53A is separated from the first contact 51A and the second contact 52A to place the switch 50A in the off state, in which the first contact 51A and the second contact 52A are not electrically connected to each other. As a result, no power is supplied from the battery 40 to the electronic component 30.

In the case of the adhesive plaster module 1A being attached to the object 300 as illustrated in FIG. 5, the movable part 53A of the switch 50A is pressed by the object 300 to move toward the substrate 10, so that the first contact 51A and the second contact 52A come in contact with the upper face of the 53A to be electrically connected to each other. As a result, power is supplied from the battery 40 to the electronic component 30 through the interconnection 71, the interconnection 74, the switch 50A, and the interconnection 72, thereby making the electronic component 30 operable.

As illustrated in FIG. 6, the upper-side packaging material 81 and the lower-side packaging material 82 may cover the adhesive plaster module 1A for protection before use. In such a case, the adhesive plaster module 1A is removed from the upper-side packaging material 81 and the lower-side packaging material 82 before the adhesive plaster module 1A is attached to and used on an object. The upper-side packaging material 81 and the lower-side packaging material 82 may be made of insulating material such as resin.

The second embodiment provides advantages in addition to the advantages of the first embodiment as follows. Without using an existing switch, a switch having a desired shape may be made in the production steps of the substrate 10.

### Third Embodiment

The third embodiment is directed to another example of the adhesive plaster module that has a switch different from the switch used in the first embodiment. In connection with the third embodiment, a description of the same or similar constituent elements as those of the previously provided descriptions may be omitted as appropriate.

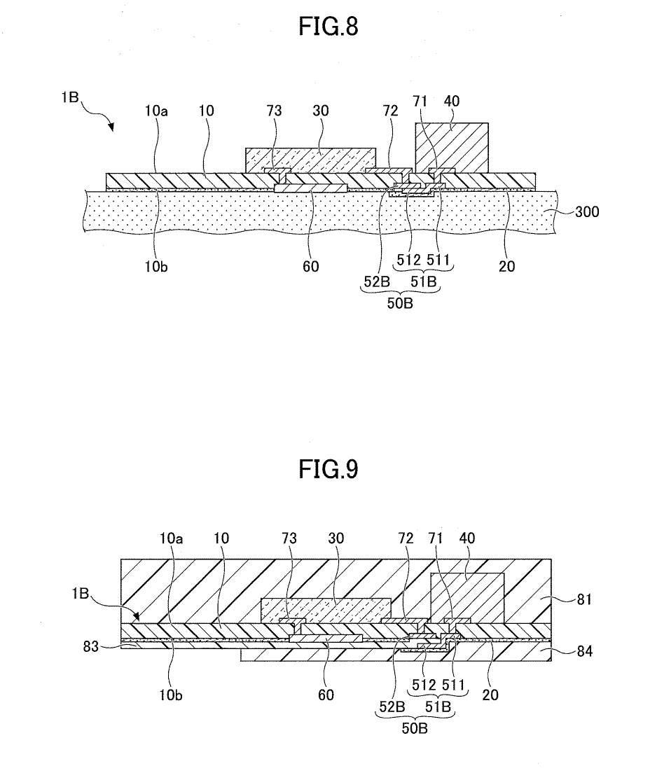

FIG. 7 is a cross-sectional view illustrating an example of an adhesive plaster module of the third embodiment. FIG. 8 is a cross-sectional view illustrating an example of the way the adhesive plaster module of the third embodiment is used. FIG. 9 is a cross-sectional view illustrating an example of the adhesive plaster module of the third embodiment that is packaged.

In FIG. 7, an adhesive plaster module 1B of the third embodiment includes the substrate 10, the adhesive layer 20, the electronic component 30, the battery 40, a switch 50B, the electrode 60, and the interconnections 71 through 73. The adhesive plaster module 1B differs from the adhesive plaster module 1 (see FIGS. 1A through 1C, for example) in that the switch 50B is provided in place of the switch 50. A further difference from the adhesive plaster module 1 (see FIGS. 1A through 1C, for example) is that the electrode 60 is partially buried in the second surface 10b of the substrate 10. It may be noted that the lower face of the electrode 60 is exposed from the second surface 10b of the substrate 10 so as to be able to come in physical contact with an object.

The switch 50B has a first contact 51B and a second contact 52B. The first contact 51B, which is partially buried in the second surface 10b of the substrate 10, is electrically connected to the positive pole of the battery 40 through the interconnection 71 inclusive of a through wire interconnect. The second contact 52B, which is partially buried in the second surface 10b of the substrate 10, is electrically connected to the positive power terminal of the electronic component through the interconnection 72 inclusive of a through wire interconnect. The first contact 513 may be made of metal having elasticity. For example, a spring may be used as the first contact 51B.

The first contact 51B has a fixed part 511 at one end thereof that is buried in the second surface 10b of the substrate 10, and has a movable part 512 at the other end thereof that extends from the fixed part 511 to project from the second surface 10b of the substrate 10 and to extend over the second contact 52B with a gap therebetween. The movable part 512 may form a step shape as illustrated in FIG. 7. The fixed part 511 and the movable part 512 may be formed seamlessly with each other as illustrated in FIG. 7, or may be formed as separate elements. The movable part 512 of the first contact 51B and the second contact 52B have faces that are opposed to each other. The opposing faces are exposed from the substrate 10, and are spaced apart from each other when not used. In the case of the adhesive plaster module 1B being not attached to an object, thus, the first contact 51B and the second contact 52B of the switch 50B are not electrically connected to each other. As a result, no power is supplied from the battery 40 to the electronic component 30.

In the case of the adhesive plaster module 1B being attached to the object 300 as illustrated in FIG. 8, the movable part 512 of the first contact 51B of the switch 50B is pressed by the object 300 to move upward (i.e., to elastically deform). As a result, the opposite faces of the movable part 512 of the first contact 51B and the second contact 52B come in physical contact with each other, so that the first contact 51B and the second contact 52B are electrically connected to each other. As a result, power is supplied from the battery 40 to the electronic component 30 through the interconnection 71, the switch 50B, and the interconnection 72, thereby making the electronic component 30 operable. It may be noted that the movable part may alternatively be formed as part of the second contact 52B.

As illustrated in FIG. 9, the upper-side packaging material 81 and lower-side packaging materials 83 and 84 may cover the adhesive plaster module 1B for protection before use. The upper-side packaging material 81 and the lower-side packaging materials 83 and 84 may be made of insulating material such as resin.

Specifically, the upper-side packaging material 81 covers the upper side of the adhesive plaster module 1B. As for the lower side of the adhesive plaster module 1B, the lower-side packaging material 83 is attached to the adhesive layer 20 to cover the electrode 60 and the surrounding areas thereof as well as the gap between the second contact 52B and the movable part 512 of the first contact 51B of the switch 50B.

Further, the lower-side packaging material 84 is attached to the adhesive layer 20 to cover the lower surface of the first contact 51B of the switch 50B on the lower side of the adhesive plaster module 1B. The lower-side packaging material 84 extends to an area where the lower face of the electrode 60 is situated, thereby partially overlapping the lower face of the lower-side packaging material 83. The lower-side packaging materials 83 and 84 are removable from the face of the adhesive layer 20.

As illustrated in FIG. 9, the lower-side packaging material 83 fills the gap between the opposite faces of the second contact 52B and the movable part 512 of the first contact 51B of the switch 50B. The lower-side packaging material 83 is an insulating material removable at the time of use of the adhesive plaster module 1B. With this arrangement, the first contact 51B and the second contact 52B are not electrically connected to each other, placing the switch 50B in the off state.

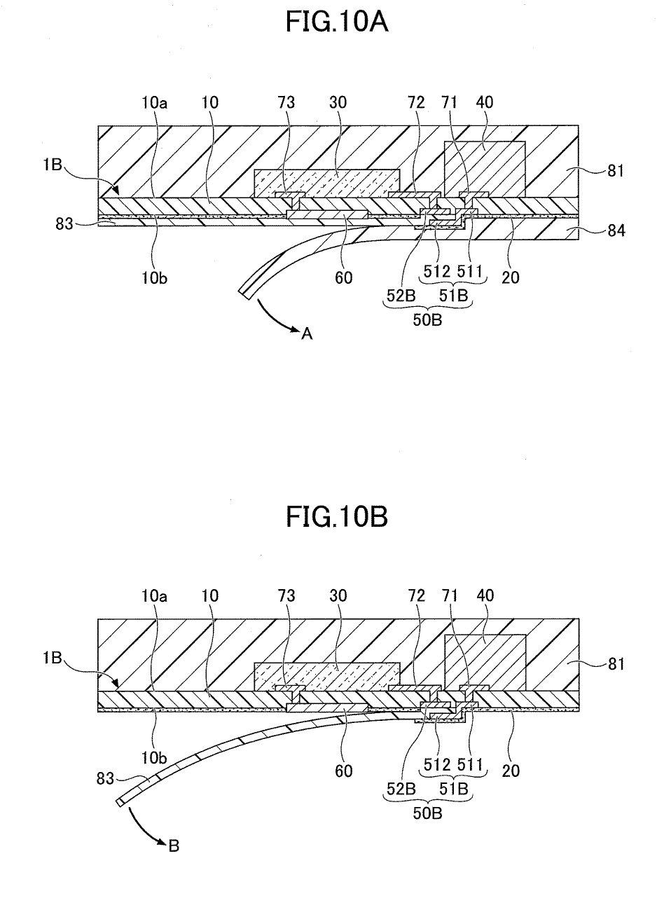

In order to use the adhesive plaster module 1B, the lower-side packaging material 84 is removed in the direction indicated by an arrow A as illustrated in FIG. 10A. The lower-side packaging material 83 is then removed in the direction indicated by an arrow B as illustrated in FIG. 10B. The adhesive plaster module 1B is in the state illustrated in FIG. 7 after these removals. Subsequently, the adhesive plaster module 1B is attached to the object 300 as illustrated in FIG. 8, which causes the switch 50B to be placed in the conductive state. Electric power is thus supplied from the battery 40 to the electronic component 30 through the interconnections 71 and 72, thereby making the electronic component 30 ready to operate.

Before the use of the adhesive plaster module 1B, the adhesive layer 20 is present on the second surface 10b of the substrate 10 as well as on the lower face of the first contact 51B. The adhesive layer 20 disposed on the lower face of the first contact 51B has an end thereof projecting further than the end of the movable part 512.

Accordingly, attaching the adhesive plaster module 1B to the object 300 causes the portion of the adhesive layer 20 projecting from the movable part 512 to adhere to the adhesive layer 20 formed on the second surface 10b of the substrate 10 as illustrated in FIG. 8 while the first contact 51B and the second contact 52B are electrically connected to each other. This arrangement prevents the switch 50B including the first contact 51B and the second contact 52B from coming in contact with the object 300, and also prevents the first contact 51B and the second contact 52B from being separated from each other.

Alternatively, the portion of the adhesive layer 20 projecting from the movable part 512 may adhere directly to the second surface 10b of the substrate 10. Namely, the area of the second surface 10b of the substrate 10 to which the portion of the adhesive layer 20 projecting from the movable part 512 adheres may have no adhesive layer 20 formed thereon.

The third embodiment provides advantages in addition to the advantages of the first embodiment as follows. Without using an existing switch, a switch having a desired shape may be made by use of a desired interconnection forming method in the production steps of the substrate 10. Further, the first contact 51B and the second contact 52B are prevented from coming in contact with the object 300, and, also, the first contact 51B and the second contact 52B electrically connected to each other are prevented from being separated from each other.

Moreover, the electrode 60 and the switch 50B situated on the side that faces the object 300 are partially buried in the substrate 10. This arrangement serves to provide a thinner adhesive plaster module 1B, and also allows a flat face to adhere to the object 300, which enables adherence without an uncomfortable tactile feel.

Further, either one of the first contact 51B and the second contact 52B can serve as a movable part. Such a simple structure can be implemented by use of simpler manufacturing steps than in the case of the structure in which a movable part is provided as a separate element.

Further, although the preferred embodiments have been described, the present invention is not limited to these embodiments, and various variations and modifications may be made without departing from the scope of the present invention.

For example, the adhesive plaster module disclosed herein is configured such that attaching the adhesive plaster module to an object causes the switch to become conductive, thereby supplying electric power from the battery to the electronic component to activate the electronic component. The scope of the invention encompasses various switch structures embodying such technological concept, and is not limited to the switch structure examples of the first through third embodiments.

It may be noted that the lower-side packaging materials 83 and 84 may be removed in a different sequence from the sequence illustrated in FIGS. 10A and 10B.

According to at least one embodiment, an adhesive plaster module is automatically powered on upon being attached to an object.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present inventions have been described in detail, it should be understood that the various changes, substitutions, and alterations could be made hereto without departing from the spirit and scope of the invention.

What is claimed is:

1. An adhesive plaster module which has an adhesive layer for adhering to an object, comprising:
a substrate having the adhesive layer;
a switch mounted on a same side of the substrate as the adhesive layer;
a battery mounted on an opposite side of the substrate from the adhesive layer; and
an electronic component mounted on the opposite side of the substrate from the adhesive layer, and configured to operate with power supplied from the battery upon the switch becoming conductive,
wherein the switch is configured to be nonconductive while the adhesive layer is not attached to the object, and configured to become conductive in response to attaching the adhesive layer to the object.

2. The adhesive plaster module as claimed in claim 1, further comprising an electrode mounted on the same side of the substrate as the adhesive layer to come in physical contact with the object,
wherein the switch includes a first contact, a second contact, and a movable part, the first contact being disposed on the same side of the substrate as the adhesive layer and electrically connected to the battery, the second contact being disposed on the same side of the substrate as the adhesive layer and electrically connected to the electronic component, the movable part being disposed to surround a perimeter of the electrode and movable along the electrode serving as a shaft, and
wherein the movable part is separated from the first contact and the second contact while the adhesive layer is not attached to the object, and moves to come in contact with the first contact and the second contact in response to attaching the adhesive layer to the object, so that the first contact and the second contact are electrically connected to each other.

3. The adhesive plaster module as claimed in claim 1, wherein the switch includes a first contact and a second contact, the first contact being disposed on the same side of the substrate as the adhesive layer and electrically connected to the battery, the second contact being disposed on the same side of the substrate as the adhesive layer and electrically connected to the electronic component,
wherein one of the first contact and the second contact has a movable part projecting from a surface of the adhesive layer, the movable part extending over the other one of the first contact and the second contact with a gap therebetween,
wherein the movable part and the other one of the first contact and the second contact have faces opposed to each other, which are separated from each other during nonuse, and
wherein the movable part moves in response to attaching the adhesive layer to the object to cause the faces to come in contact with each other, so that the first contact and the second contact are electrically connected to each other.

4. The adhesive plaster module as claimed in claim 3, wherein the adhesive layer extends on a face of the movable part facing away from the substrate, and has an end thereof projecting further than an end of the movable part.

5. The adhesive plaster module as claimed in claim 3, further comprising an insulating material filling a gap between the faces and removable at a time of use.

6. The adhesive plaster module as claimed in claim 4, wherein the movable part moves in response to attaching the adhesive layer to the object to cause the faces to come in contact with each other, and the end of the adhesive layer projecting from the end of the movable part adheres to another portion of the adhesive layer when the first contact and the second contact are electrically connected to each other.

7. The adhesive plaster module as claimed in claim 1, wherein the substrate is flexible, stretchable, and elongated.

8. The adhesive plaster module as claimed in claim 1, wherein the electronic component includes a sensor configured to acquire biological information from the object.

9. The adhesive plaster module as claimed in claim 8, wherein the electronic component includes at least one of a memory configured to store the biological information and a wireless transmission unit configured to transmit the biological information wirelessly.

10. The adhesive plaster module as claimed in claim 8, wherein the biological information includes at least one of bioelectric potential, body temperature, heart rate, arterial blood oxygen saturation, and blood glucose level.

11. An adhesive plaster module, comprising:
a substrate having an adhesive layer for adhering to an object;
a switch mounted on the substrate and configured to be turned on by a force applied to the switch, the force being generated by adherence of the adhesive layer to the object;
a battery mounted on the substrate; and
an electronic component mounted on the substrate and configured to receive power from the battery through the switch that is turned on.
